(19) United States
(12) Patent Application Publication
(10) Pub. No.: US 2016/0106591 A1
(43) Pub. Date: Apr. 21, 2016

(54) METHODS FOR PREVENTING OR TREATING POSTERIOR CAPSULAR OPACIFICATION

(71) Applicant: NEXXTVISION LLC, Naperville, IL (US)

(72) Inventor: George J. McArdle, Naperville, IL (US)

(21) Appl. No.: 14/892,174

(22) PCT Filed: Apr. 21, 2014
(86) PCT No.: PCT/US14/34782

§ 371 Date: Nov. 18, 2015

Related U.S. Application Data
(60) Provisional application No. 61/814,056, filed on Apr. 19, 2013.
(60) Provisional application No. 61/846,357, filed on Jul. 15, 2013.

(51) Int. Cl. A61F 9/008; A61F 2/16

(52) U.S. Cl. CPC A61F 9/00834; A61F 2/1602; A61F 9/00827; A61F 9/0084; A61F 2009/00853; A61F 2009/0087; A61F 2009/00889

(57) ABSTRACT

The present invention relates to methods and apparatus for preventing or treating posterior capsular opacification in a subject in need of prophylaxis or treatment for posterior capsular opacification, including a subject undergoing cataract surgery by ablating epithelial cells on an interior surface of the lens capsule with a multi-photon laser system.

### CROSS-REFERENCE TO RELATED APPLICATIONS

This international application claims priority to U.S. Application No. 61/814,056 filed on Apr. 19, 2013 and to U.S. Application No. 61/846,357 filed on Jul. 15, 2013, both of which are incorporated herein by reference in their entirety.

### FIELD OF THE INVENTION

The present invention relates to methods and apparatus for preventing or treating posterior capsular opacification.

### BACKGROUND OF THE INVENTION

Cataract surgery generally involves removing the cataractous lens by a surgical procedure and replacing it with an artificial intraocular lens within the lens capsule. The artificial intraocular lens is inserted into the lens capsule through an incision in the anterior portion of the lens capsule, while the posterior surface is left intact as an envelope or bag, and the lens capsule itself remains attached through the zonules. The artificial intraocular lens will ordinarily remain in place throughout the patient's life.

Posterior capsular opacification (PCO) is one of the most frequent complications of cataract surgery. Also known as secondary cataract, it occurs in a high percentage of patients and can occur months or years after the cataract surgery. In the past decade, results from a number of experimental and clinical studies have led to a better understanding of the pathogenesis of PCO. Residual lens epithelial cells are often left behind after cataract surgery. These residual epithelial cells seek to migrate along the back surface of the implanted artificial intraocular lens and opacify the interior posterior surface of the lens capsule. These residual epithelial cells may proliferate and increase the degree of opacification.

Current treatments for posterior capsular opacification have employed the use of various surgical instruments and the application of physical, biochemical, laser, and other techniques. For example, capsulotomy using a YAG laser may be the most common treatment. The YAG laser is used to create an opening in the center of the posterior capsule, to produce a clear area for light to reach the retina. In other words, the YAG laser is used to open a hole in the posterior capsule where the opacification from the epithelial cells occurs. The hole is not so large as to allow the intraocular lens to fall into the back of the eye, but it is of sufficient size to clear the visual access. This is known as a YAG capsulotomy. Although this procedure is non-invasive, complications such as retinal detachment, lens damage, glaucoma, and macular edema may arise.

Allan US20040047900 states that posterior capsular opacification is inhibited by administration of a polymer, having a ligand for a death receptor immobilized on the surface, preferably joined by a spacer into the lens capsule following cataract surgery. The ligand is preferably a Fas ligand. A preferred spacer is polyethylene glycol. The polymer preferably constitutes an intraocular lens.

Zhang US20070129286 states that PCO can be prevented by rapidly and selectively inducing detachment and/or cell death of lens epithelial cells without significantly damaging other ocular cells and tissues. PCO prevention is accomplished via application of treatment solution or solutions. The treatment solution is applied or introduced into the lens capsular bag before, during, or after cataract surgery. The treatment solution comprises an ion transport mechanism interference agent, which either alone or in combination with other treatment agents such as an osmotic stress agent and an agent to establish a suitable pH, selectively induces detachment and/or death of lens epithelial cells such that posterior capsular opacification is prevented. The treatment solution selectively induces cellular death and/or detachment of lens epithelial cells while other ocular cells and tissue remain substantially unharmed and without lengthy preoperative pre-treatment.

Schuele et al. US20110172649 discusses a system for ophthalmic surgery, comprising a laser source configured to deliver a laser beam comprising a plurality of laser pulses having a wavelength between about 320 nanometers and about 430 nanometers and a pulse duration between about 1 picosecond and about 100 nanoseconds; and an optical system operatively coupled to the laser source and configured to focus and direct the laser beam in a pattern into one or more intraocular targets within an eye of a patient, such that interaction between the one or more targets and the laser pulses is characterized by linear absorption enhanced photodecomposition without formation of a plasma or associated cavitation event.

Larsen US20100292676 discusses a method and a system for non- or minimally disruptive photomanipulation of the lens and/or its constituents collectively or selectively of an animal or human eye. It states that photonic excitation of specific molecular constituents of the human eye using blue light or ultraviolet is problematic because the energetic photons cause damage to the cornea and the living layers of the lens. Additional problems include retinotoxicity and poor penetration of cataractous lenses. A method of circumventing this problem is to use multiphoton excitation. Two-photon excitation achieves specific electronic excitation by laser light with a high intensity and half the wavelength required to induce the desired effect by means of a single photon.

Pollhammer et al., “In situ ablation of lens epithelial cells in porcine eyes with the laser photolysis system,” Journal of Cataract & Refractive Surgery, Volume 33, Issue 4 , Pages 697-701, April 2007, discusses an in vitro study on the ablation of lens epithelial cells on the anterior portion of a porcine lens capsule. Pollhammer et al. state that their method could provide success for intraoperative prophylaxis of PCO in patients. However the teachings of Pollhammer et al. are not transferable to a clinical method without substantial modifications and a great deal of experimentation. For one thing, the epithelial cells on the porcine lens capsules could not have been seen in vivo, so the authors used extracted lenses. The confocal microscope used by the authors would not have provided a clinically acceptable degree of resolution. Furthermore, the Pollhammer procedure relies on the use of a Q-switched Nd: YAG laser, which would be likely to damage the lens capsule or anterior tissues if it were used to ablate the epithelial cells found in PCO in vivo. Indeed, the YAG laser device is the same as used for phacoemulsification (breaking up the native crystalline lens) during microincision cataract surgery.

Ramasamy et al., “Multiphoton imaging and laser ablation of rodent spermatic cord nerves: potential treatment for patients with chronic orchialgia”, J. Urol. 2012 February; 187(2):733-8, discusses multiphoton microscopy, a novel laser imaging technology, to identify and selectively ablate spermatic cord nerves in the rat.

Gualda et al., “Femtosecond infrared intrastromal ablation and backscattering-mode adaptive-optics multiphoton microscopy in chicken corneas,” Biomedical Optics Express, Vol. 2, Issue 11, pp. 2950-2960 (2011), discusses the performance of femtosecond laser intrastromal ablation with backscattering-mode adaptive-optics multiphoton microscopy in ex vivo chicken corneas.

McArdle et al. US20110160622 generally relates to an apparatus and processes for preventing or delaying presbyopia by ablating epithelial cells in the germinative zone or the pregerminative zone of the crystalline lens of the eye so that onset or progression of presbyopia or one or more symptoms is delayed or prevented. The disclosure also relates to processes and apparatus for promoting formation of suture lines in the crystalline lens of the eye so that onset or progression of presbyopia or one or more symptoms is delayed or prevented. The present disclosure also relates to processes and apparatus for creating disruptions in the vitreous humor of the eye.

It is believed that multi-photon lasers have not been used previously for clinical treatments in general, nor for ophthalmic surgery in particular.

Despite the existence of treatments for PCO, the search in the field of PCO remains open to the identification of improved treatments or preventative approaches, particularly ones that will avoid the need for additional ophthalmic surgery on the patient.

### BRIEF SUMMARY OF THE INVENTION

The present disclosure relates to methods and apparatus for preventing or treating posterior capsular opacification in a subject in need of prophylaxis or treatment for posterior capsular opacification, including a subject undergoing cataract surgery. The subject has a biconvex lens capsule that has an anterior portion and a posterior portion, and each of those portions has an exterior surface and an interior surface. The methods comprise ablating epithelial cells at an interior surface of the lens capsule with a multi-photon laser system. The apparatus can include an imaging system for imaging epithelial cells at an interior surface of a lens capsule, preferably beneath the anterior capsule, prior to or concomitantly with cataract surgery. The imaging system can be a multi-photon laser system, or another imaging system capable of identifying epithelial cells.

As one aspect of the present invention, methods for preventing or treating posterior capsular opacification in a subject in need of prophylaxis or treatment of posterior capsular opacification are provided. The subject has a lens capsule has an anterior portion and a posterior portion, and each of the anterior and posterior portions has an exterior surface and an interior surface. The methods comprise imaging epithelial cells on one or more of interior surfaces of the lens capsule, preferably beneath the interior surface of the anterior capsule, using an imaging system or technique selected from the group consisting of confocal microscopy, adaptive optics ultrasound, detecting a chemical agent and/or a biological agent, and multi-photon laser imaging, and combinations thereof. The methods can also include using the imaging system or technique to ablate the epithelial cells. The ablating system generally includes a femtosecond laser as the laser source and generates one or more beams that are focused at a point of ablation at or beneath an interior surface of the lens capsule. It is desired that the epithelial cells are ablated without damaging or breaking the lens capsule. The methods can also include the steps of removing a cataractous lens from the subject while retaining the lens capsule, and inserting an artificial intraocular lens. Either before and/or after the removing step (preferably before), the epithelial cells are imaged at one or both of the interior surfaces of the lens capsule. Alternatively or additionally, the epithelial cells can be imaged at the interior surface of the anterior portion of the lens capsule before and/or after the inserting step, and the imaged epithelial cells are ablated before or after (preferably before) the inserting step, before and/or after the artificial intraocular lens is inserted in the lens capsule. This could be used for prophylaxis of PCO, by ablating epithelial cells before they can migrate to the posterior of the lens capsule and cause opacification. As another embodiment, the methods can include diagnosing the subject as having posterior capsular opacification perhaps some months after the subject has had cataract surgery, and the step of imaging epithelial cells comprises imaging the epithelial cells at the interior surface of the posterior portion of the lens capsule using the imaging system. This could be used for treating of PCO caused by epithelial cells at the interior surface of the posterior portion of the lens capsule.

As another aspect of the present invention, an apparatus for preventing or treating posterior capsular opacification is provided. The apparatus comprises an imaging system adapted for imaging epithelial cells at or beneath an anterior capsule of a crystalline lens, the imaging system being selected from the group consisting of a confocal microscope system, an adaptive optics system, an ultrasound system, a chemical agent detection system, a biological agent detection system, a multi-photon laser system, and combinations thereof. The apparatus also comprises an ablation system comprising a laser source having sufficient energy to ablate epithelial cells beneath a lens capsule without rupturing the capsule. The laser source can be a femtosecond laser. Preferably the imaging system and the ablation system are adapted so as not to damage the cornea, such as by having a laser source provides a laser beam having a power, laser wavelengths and/or pulse duration that does not damage the cornea.

Optionally a single laser system can be provided which is adapted for both imaging and ablating epithelial cells at an interior surface of a lens capsule. Such an apparatus can comprise an imaging mode and an ablation mode which differ in terms of power, laser wavelengths and/or pulse durations. The apparatus comprises a laser system that does not damage the cornea.

As yet another aspect of the present invention, a method is provided for preventing posterior capsular opacification in a subject in conjunction with cataract surgery on the subject. The method comprises imaging epithelial cells on one or more interior surfaces of the lens capsule before or concomitantly with cataract surgery on the subject. The method also comprises ablating the imaged epithelial cells, wherein the ablating occurs prior to cataract surgery, prior to incising the lens capsule for cataract surgery, prior to or after inserting an artificial intraocular lens, or prior to a diagnosis of posterior capsular opacification. Preferably the epithelial cells are imaged using an imaging technique selected from the group consisting of confocal microscopy, optical coherence tomography, adaptive optics, ultrasound, detecting a chemical agent and/or a biological agent, multi-photon laser imaging, and combinations thereof. Preferably the method comprises imaging the epithelial cells on the interior surface of the anterior of the lens capsule before and/or after removing the cataractous lens and before and/or after inserting the artificial intraocular lens.

### DETAILED DESCRIPTION OF THE INVENTION

The crystalline lens of the eye has a generally circular cross-section having two convex refracting surfaces. The crystalline lens is suspended by a circular assembly of collagenous fibers called zonules, which are attached at their inner ends to the lens capsule and at their outer ends to the ciliary body, a muscular ring of tissue located just within the outer supporting structure of the eye, the sclera. The crystalline lens is a transparent, biconvex membrane with an anterior portion that is less spherical than the posterior portion. The core of the crystalline lens comprises a nucleus of primary lens fibers which are elongated along the visual axis. The core is surrounded by a cortex of elongated secondary lens fibers. At the anterior face of the lens resides a layer of cuboidal cells which make up the central zone of the lens. An anterior monolayer serves as the germ cell layer of the lens, a stratified epithelia-like tissue. The lens capsule covers the crystalline lens, including the epithelial cells and the primary and secondary lens fibers. The lens capsule is a clear, membrane-like structure that is relatively elastic. The membrane of the lens capsule has an anterior portion and a posterior portion, and each of those has an exterior surface which faces the other structures of the eye, and an interior surface which faces the crystalline lens.

A cataractous lens is a crystalline lens having a cataract, which is any form of opacity in the lens which interferes with light passage through the lens. As a result, a person with a cataractous lens will have impaired vision. Cataract surgery involves the removal of the natural crystalline lens and replacement with an artificial intraocular lens within the natural lens capsule. This is typically done by making a small incision in the anterior portion of the lens by capsulorhexis. The lens is then dissolved by phacoemulsification and removed. The artificial intraocular lens is inserted through the small incision and then deployed, such as by unfolding and/or attaching haptics.

The present methods can be prophylactic against PCO (that is, prevent PCO or reduce its likelihood of occurring) by ablating epithelial cells at the anterior portion of the lens capsule of the crystalline lens before and/or concomitantly with cataract surgery. The epithelial cells at the anterior portion of the lens capsule are structurally part of the crystalline lens rather than the lens capsule though they are in contact with the lens capsule. In other words, before, during or after removal of the cataractous lens by phacoemulsification or another technique, the present methods are used to image and ablate any epithelial cells on the crystalline lens or remaining after removal of the lens, particular at the interior surface of the anterior portion of the lens capsule.

The present methods can include ablating epithelial cells at the posterior portion of the lens capsule and/or at the posterior of the intraocular lens after and apart from cataract surgery, after posterior capsular opacification has been diagnosed. The epithelial cells at the posterior portion may be on the lens capsule itself, or near to it, or attached to the intraocular lens that faces the posterior portion of the lens capsule. The ablation may be performed at least one month after the cataract surgery, alternatively at least two months, alternatively at least six months, alternatively at least one year, alternatively at least two years, after the subject has had cataract surgery.

In the present methods, the step of imaging epithelial cells on one or more of the interior surfaces of the lens capsule uses an imaging technique selected from the group consisting of confocal microscopy, adaptive optics, ultrasound, detecting a chemical agent and/or a biological agent, multi-photon laser imaging, and combinations thereof. In the present apparatus, an imaging system is provided for imaging epithelial cells at the lens capsule. The imaging system can be selected from the group consisting of a confocal microscope system, an adaptive optics system, an ultrasound system, a chemical agent detection system, a biological agent detection system, and a multi-photon laser system, and combinations thereof

There are various types of confocal microscope systems and techniques: Confocal laser scanning microscopes use multiple mirrors (typically 2 or 3 scanning linearly along the x and the y axis) to scan the laser across the sample and descan the image across a fixed pinhole and detector. Spinning-disk confocal microscopes use a series of moving pinholes on a disc to scan spot of light. Programmable Array Microscopes use an electronically controlled spatial light modulator that produces a set of moving pinholes. By scanning over a surface or an area, the confocal laser systems are adapted to provide two-dimensional or three-dimensional imaging. Such systems can be used to image such epithelial cells.

In a photoacoustic system or technique, energy delivered to the area of the lens capsule, for example by laser, will be absorbed and converted into heat, leading to transient thermoplastic expansion and thus ultrasonic emission. The generated ultrasonic waves are then detected by ultrasonic transducers to form images. It is known than optical absorption is closely associated with physiologic properties, such as hemoglobin concentration and oxygen saturation. As a result, the magnitude of the ultrasonic emission that is proportional to the local energy deposition reveals physiological specific optical absorption contrast. Two- or three-dimensional images of the targeted tissues can then be formed. If the laser pulse is short enough (such as by use of a Nd-YAG), a local acoustic effect is generated that can be imaged by an ultrasonic transducer in 2D or 3D format. Because photoacoustic and ultrasonic imaging can share the same array and receiver, the image produced by them can simultaneously provide information on the thermal and anatomical structure, and location of the tissue in a rapid succession such as real time (video) images. U.S. Pat. No. 7,964,214 (Peyman) discloses a photoacoustic system that may be employed in the present methods and apparatus.

In an adaptive optics system, aberrations in light are compensated by measuring the distortions in a wavelength and using a deformable mirror to correct them. The aberrations are caused by light passing through ocular structures. Such a system could be used to improve resolution and clarity of focus, and adaptive optics can be used in conjunction with other systems such as optical coherence tomography. After imaging one section, the mirrors are rotated or otherwise adjusted. Adaptive optics provides high accuracy with very low resolution.

The imaging system or technique can include a chemical or biological agent attached to a detectable label and/or a detector for detecting such a label. Such agents can include dyes that preferentially dye epithelial cells or cell surfaces. The dyed or labeled cells can be visualized using a microscope or other detector. As an example of a biological agent, an antibody that specifically binds to an epitope on an epithelial cells (see, for example, Gioanni, J. et al., “Charaterization of a New Surface Epitope Specific for Human Epithelial Cells Defined by a Monoclonal Antibody and Application to Tumor Diagnosis”, Cancer Research, vol. 47, pp. 4417-4424 (1987)), can be conjugated to a dye or label to provide for imaging of the epithelial cells. An example of a monoclonal antibody is CALAM 27, which is directed to surface epitopes of both normal and malignant epithelial cells. As another example, EpCAM (CD326) is a surface protein on epithelial cells, and a fluoresently-labeled antibody or other targeting moiety can be directed to EpCAM and used to image epithelial cells. Antibodies or targeting moieties to other epithelial cell specific markers are also contemplated.

Preferably, the imaging system or technique is a multi-photon laser system used to locate the epithelial cells, and a femtosecond laser is used as the laser source for the multi-photon laser system for imaging and optionally for ablating the epithelial cells. The components of known multi-photon microscopes can be used in the multi-photon laser system contemplated herein. Multi-photon microscopes are available from a number of commercial sources. An example of a multi-photon laser microscope is the A1R-MP from Nikon, described in the brochure entitled “MR MP Multiphoton confocal microscope”, copyright 2009 Nikon Corporation, which is incorporated by reference herein. Multi-photon microscopy is an imaging technique that routinely allows imaging of living tissue up to a depth up of about one millimeter, though imaging at greater depths can be done. In two-photon microscopy, two photons of the laser light are absorbed by the object to be imaged, thereby resulting in excitation. Due to the multi-photon absorption the background signal is suppressed. Two-photon excitation can be a superior alternative to confocal microscopy due to its deeper tissue penetration, efficient light detection and reduced phototoxicity. Preferably the multi-photon laser system comprises an optic that allows imaging deeper in the eye with adequate resolution (for example, resolution of individual cells). For example, it is contemplated that multi-photon laser system comprises allows imaging at a depth of more than 0.5 mm from the tissue surface, for example at a depth of 3.5 mm or more, preferably at a depth of 5 mm or more.

Because the multi-photon laser microscopy is a non-linear process, it does not cause photo-bleaching anterior and posterior to the imaged tissue, in contrast to a linear process. The use of multiphoton excitation avoids this problem. Two-photon excitation achieves specific electronic excitation by laser light with a high intensity and half the wavelength required to induce the desired effect by means of a single photon. The high intensity of the light increases the probability of exciting the fluorescence by a two-step process, where the molecule is first excited to a virtual level by the first photon and subsequently by another photon that strikes the electron within the lifetime of the fluorescent state.

Multi-photon laser systems are known in the industry, and its various components are generally known. An example of two-photon laser microscopy system is found in Denk, et al. U.S. Pat. No. 5,034,613 which discloses a laser scanning microscope that produces molecular excitation in a target material by simultaneous absorption of two photons to thereby provide intrinsic three-dimensional resolution. Another example of a multi-photon laser system is found in Schnitzer US20040260148, which includes a pulsed laser, a pre-compensator for chromatic dispersion, a transmission optical fiber, and a graded refractive index lens. Other examples of multi-photon laser systems are set forth in the Ramasamy and Gualda publications discussed above. In the present methods and apparatus, the multi-photon laser system is adapted to receive laser energy from a laser source (or, it includes some type of laser light receiver). The multi-photon laser system can include a beam splitter and an objective lens optically coupled to the multi-photon excitation light source and configured to focus the separate beams of laser light to an ablation point. The multi-photon laser system can include detectors, sensors, mirrors, lenses, modulators, polarizers, and other components.

Multi-photon microscopes are frequently used with fluorescent materials such as a dye, that is, materials that will fluorescence after absorbing multiple photons (rather than just one photon). For example, a dye requiring an excitation wavelength of 400 nm will be illuminated by a laser source operating at 800 nm such that single photon excitation does not occur in the specimen since the dye does not absorb light at 800 nm. Use of a pulsed high-power excitation laser (such as a femtosecond laser) provides a sufficiently high photon density at the point of focus for at least two photons to be absorbed (essentially simultaneously) by the fluorescent material. It is contemplated that a suitable fluorescent material can be administered to the interior of the lens capsule of the subject prior to the imaging step. It is also contemplated that the epithelial cells may be capable of auto-fluorescence under some conditions and/or may be imaged using third harmonic generation of the laser source, and the administration of a fluorescent material would be optional or would not be performed.

The present methods and apparatus provide non-invasive techniques for preventing or treating PCO. It is greatly advantageous that the epithelial cells causing PCO can be removed and PCO can be treated without an incision to the lens capsule and/or other structures of the eye such as the cornea. Locating the epithelial cells can merely be imaging non-capsular material on an interior surface of the lens capsule (most likely the posterior interior surface if PCO has been diagnosed), or can be identifying epithelial cells as such. Locating can include providing an exact location of epithelial cells or an approximate location.

In one aspect of the present invention, a method for preventing posterior capsular opacification in a subject is provided. The method is employed in conjunction (that is, just before or concomitantly with) cataract surgery on the subject. The method comprises imaging epithelial cells on one or more of interior surfaces of the lens capsule before or concomitantly with the cataract surgery on the subject. The method also comprises ablating the imaged epithelial cells, wherein the ablating occurs prior to cataract surgery, prior to capsulorhexis, prior to emulsifying or removing the natural crystalline lens, prior to inserting the artificial crystalline lens, or prior to a diagnosis of posterior capsular opacification. The imaging step or technique can be selected from the group consisting of confocal microscopy, ultrasound, detecting a chemical agent and/or a biological agent, multi-photon laser imaging, or another imaging technique. For example, in this aspect of the present invention, Optical Coherence Tomography can be employed for imaging the epithelial cells.

Optical Coherence Tomography (OCT) is a technique for obtaining sub-surface images of tissues such as cell masses at a resolution equivalent to a low-power microscope. An optical beam is directed at the tissue, and a small portion of this light that reflects from sub-surface features is collected. Most light is not reflected but scatters off at large angles. OCT uses interferometry to record the optical path length of received photons allowing rejection of most photons that scatter multiple times before detection. Thus OCT can build up clear 3D images of thick samples by rejecting background signal while collecting light directly reflected from surfaces of interest. OCT provides tissue morphology imagery at much higher resolution (at a level up to or better than 10 μm) than other imaging techniques such as MRI or ultrasound.

The imaging system allows the user to image epithelial cells at the lens capsule and target them for ablation. Targeting can include locating, viewing, or identifying the cells, and selecting them for ablation. Targeting can be done manually or by automation, such as with a software module that processes data from the imaging system and provides instructions to the ablation system for positioning the ablation point of the laser or ablation beam. The present methods and apparatus can include a computer or other processor to run the software module or otherwise control the laser source and/or the multi-photon laser system.

A femtosecond laser can be used for ablation and in the multi-photon laser system of the present methods and apparatus. In contrast to the photo-ablative ultraviolet lasers, femtosecond laser pulses in the near infrared or visible range can pass through transparent corneal tissue without significant one-photon absorption. Only when pulses are focused at a point is the intensity of the beam sufficient to cause nonlinear, typically, multi-photon absorption. Because the absorption is nonlinear, the laser-affected region tends to be highly localized, leaving the surrounding region unaffected, or minimally affected.

Epithelial cells of the crystalline lens can be ablated by any suitable technique, but will generally be ablated using a femtosecond laser-based surgical technique. Ablating cells means removing cells, including by cutting, extirpating, vaporizing, abrading, or any other suitable technique for removing cells from a living tissue. When using a laser-based surgical technique, ablated cells are usually vaporized.

Accordingly, in a preferred embodiment the ablation laser beam originates from a laser system comprising at least one ultra fast laser to enable multi-photon effect, such as two-photon effect. The laser source provides light at a wavelength or about 800 nm or higher, preferably 1030 nm. By use of third harmonic generation, light with a wavelength of 343 nm can be created from the laser source having a wavelength of 1030 nm. Preferably the treatment laser system emits laser light in the wavelength range 200-1500 nm, preferably in the range 300-550nm, in the range 550-700 nm, in the range 700-1000 nm, in the range 1000-1500 nm. In a preferred embodiment the treatment laser beam originates from a Titanium-sapphire laser emitting at 800 nm or a band or line within +/−300 nm of 800 nm. It is preferred that the abalation laser beam has a tolerance less than 10 microns, alternatively less than 5 microns, in order to provide the precision desired for ablating the epithelial cells at or beneath the anterior capsule.

Multi-photon lasers have been used for imaging; it is believed that the present disclosure is the first to provide an in vivo clinical treatment using a multi-photon laser. In the present methods and apparatus, it is preferred that the imaging system has resolution to the cellular level, or approximately 10 microns and below.

The present apparatus may include a laser source which provides laser light in pulses. The laser source may include a laser-generating element that produces pulses of laser light having a selected pulse length and/or pulse rate. The pulse length and pulse rate are selected in conjunction with laser wavelength and energy level so that the application of laser light provides imaging or ablation of the desired epithelial cells without unduly damaging surrounding tissue or the cornea. Any suitable pulse length may be employed in the present processes and apparatus. Laser light may be applied to the crystalline lens in pulse(s) having a length on the order of nanoseconds, for example, tens or hundreds of nanoseconds. Alternatively, the pulse length may be on the order of microseconds, picoseconds, or femtoseconds. With a femtosecond laser, each pulse of laser light has a pulse length on the order of femtoseconds (or quadrillionths of a second).

Short pulse lengths are desirable to avoid transferring heat or shock to material being lasered, which means that ablation can be performed with virtually no damage to surrounding tissue. Further, a femtosecond laser can be used with extreme precision. Femtosecond pulse generating lasers are known to the art. Lasers of this type are capable of generating pulse lengths presently as short as 5 femtoseconds with pulse frequencies presently as high as 10 KHz.

While it is currently preferred that a femtosecond laser and the multi-photon laser system are used for ablation, since their use will reduce or minimize collateral damage, other lasers are also contemplated for the present methods and apparatus, provided they do not produce excessive collateral damage. Moreover, others source of ablation may be viable including focused ultrasound or other focused energy on the electromagnetic spectrum.

Multi-photon laser microscopy for imaging and targeting is preferred for the present methods and apparatus because it allows precise targeting of the epithelial cells in the interior of the lens capsule or beneath the anterior capsule without causing damage to the other structure or tissues. In a multi-photon laser, the beam is split, and the beam reconnects at the target point. The multi-photon laser system is non-linear, which provides advantages over linear laser processes where the full laser energy passes through tissue on the way to the target. Since the lifetime of the virtual level is very short, a second photon should be available within very short time—hence the high intensity. On the other hand, the pulse energy should be kept relatively low to avoid thermal or chemical damage to the surrounding tissue. Accordingly, the light is preferably pulsed so that the requirement of high intensity may be fulfilled through a high peak-intensity. A high peak power, but low energy pulse is obtained by using a picosecond, nanosecond or femtosecond laser and by focusing the laser light into the region of the tissue of interest. For example, pulse durations of 100 nanoseconds or less, alternatively about 10 nanoseconds or less, alternatively about 1 nanosecond or less, alternatively about 100 picoseconds or less, alternatively about 1 picosecond or less, alternatively about 100 femtoseconds or less, alternatively about 10 femtoseconds or less are contemplated. The combination of focusing and two-photon excitation significantly reduces the risk of damage in the surrounding tissue because the flux of energy needed to achieve excitation exists only at the focal point. For imaging, it is contemplated that the laser energy may be between about 15 nJ and 25 nJ, alternatively between about 18 nJ and about 22 nJ. Suitable laser repetition rates for imagining may include from about 8 MHz to about 12 MHz, alternatively about 10 MHz. In an ablation mode, it is contemplated that the epithelial cells will ablated with laser light having an energy in the range of from about 35 nJ to about 45 nJ, alternatively from about 37 nJ to about 42 nJ.

The imaging step and the ablation step can be performed by a single system in the present methods and apparatus or there can be separate systems. Preferably the present methods and apparatus employ a single laser source and multi-photon laser system both for imaging and for ablation. The apparatus can include a switch for increasing the power to a level that will cause ablation. A multi-photon laser can include a switch to change from ablation and imaging modes. Alternatively, a combined system can operate by increasing the energy level of the laser to a higher level, so that it causes ablation of epithelial cells rather than excitation for imaging.

The present methods and apparatus can also include a patient interface such as a docking system or means that is adapted to contact the eye of the subject and hold it substantially steady as the eye undergoes treatment. Suitable patient interfaces and docking systems have been developed and used in other ophthalmic surgical procedures, such as LASIK and cataract surgery. Examples of such docking systems are described at Raksi et al. US20120283708 and Juhasz et al. US20130050649. The present methods and apparatus can also include a tracking system or means which will adjust the ablating laser for minor movements of the eye during the present procedure. Suitable tracking systems are already known in the art. Examples of such tracking systems are described at Frey et al. US20020013577, Zepkin et al. US20030225398, and Spooner US20120078240.

All patents and publications identified herein are incorporated by reference in their entireties.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method for preventing or treating posterior capsular opacification in a subject in need of prophylaxis or treatment of posterior capsular opacification, wherein the subject has a lens capsule having an anterior portion and a posterior portion, and each of the anterior and posterior portions has an exterior surface and an interior surface, the method comprising:
imaging epithelial cells on one or more interior surfaces of the lens capsule using an imaging technique selected from the group consisting of confocal microscopy, adaptive optics, ultrasound, detecting a chemical agent and/or a biological agent, multi-photon laser imaging, and combinations thereof; and
ablating the imaged epithelial cells.

2. The method of claim 1, wherein a laser is used for imaging the epithelial cells, and the same laser used for imaging is used for ablating the epithelial cells.

3. The method of claim 1, wherein the ablation system comprises a femtosecond laser as the laser source.

4. The method of claim 1, wherein the epithelial cells are ablated without damaging or breaking the lens capsule.

5. The method of claim 1, wherein the method further comprises removing a cataractous lens from the subject while retaining the lens capsule, and inserting an artificial intraocular lens; and either before and/or after the inserting step, imaging and ablating the epithelial cells one or both of the interior surfaces of the lens capsule.

6. The method of claim 5, wherein the epithelial cells are imaged at the interior surface of the anterior portion of the lens capsule before the inserting step.

7. The method of claim 6, wherein the imaged epithelial cells are ablated before the inserting step, when the artificial intraocular lens is in the lens capsule.

8. The method of claim 5, wherein the method further comprises diagnosing the subject as having posterior capsular opacification at least one month after the subject has had cataract surgery, and the step of imaging epithelial cells comprises imaging the epithelial cells at the interior surface of the posterior portion of the lens capsule using the multi-photon laser system.

9. A method for preventing posterior capsular opacification in a subject in conjunction with cataract surgery on the subject, wherein the subject has a lens capsule having an anterior portion and a posterior portion, and each of the anterior and posterior portions has an exterior surface and an interior surface, the method comprising:
imaging epithelial cells on one or more of interior surfaces of the lens capsule before or concomitantly with cataract surgery on the subject; and
ablating the imaged epithelial cells.

10. The method of claim 9, wherein the epithelial cells are imaged using an imaging technique selected from the group consisting of confocal microscopy, optical coherence tomography, adaptive optics, ultrasound, detecting a chemical agent and/or a biological agent, multi-photon laser imaging, and combinations thereof.

11. The method of claim 9, wherein the method further comprises removing a cataractous lens from the subject while retaining the lens capsule, and inserting an artificial intraocular lens; and either before and/or after the inserting step, but during the cataract surgery, imaging the epithelial cells on one or both of the interior surfaces of the lens capsule.

12. The method of claim 11, wherein the method comprises imaging the epithelial cells on the interior surface of the anterior of the lens capsule after removing the cataractous lens and before inserting the artificial intraocular lens.

13. An apparatus for preventing or treating posterior capsular opacification, the apparatus comprising:
an imaging system adapted for imaging epithelial cells at or beneath an anterior capsule of a crystalline lens, the imaging system being selected from the group consisting of a confocal microscope system, an adaptive optics system, an ultrasound system, a chemical agent detection system, a biological agent detection system, a multi-photon laser system, and combinations thereof; and
an ablation system comprising a laser source having sufficient energy to ablate epithelial cells beneath a lens capsule without rupturing the capsule.

14. The apparatus of claim 13, wherein the imaging system is a multi-photon laser system operatively connected to a laser source and adapted for receiving the radiation from the laser source and generating a plurality of laser beams that converge at a focal point.

15. The apparatus of claim 13, wherein the laser source is a femtosecond laser.

16. The apparatus of claim 13, wherein the laser source provides a laser beam having a power, laser wavelengths and/or pulse duration that does not damage the cornea.
